(19) United States
(12) Patent Application Publication
(10) Pub. No.: US 2010/0196462 A1
(43) Pub. Date: Aug. 5, 2010

(54) Method for Manufacturing a Pharmaceutical Form of Oseltamivir Phosphate

(76) Inventor: Sebastien Bardot, Orleans (FR)

Correspondence Address: OLIFF & BERRIDGE, PLC, P.O. BOX 320850, ALEXANDRIA, VA, 22320-4850, US

(21) Appl. No.: 12/087,455

(22) PCT Filed: Jan. 11, 2007
(86) PCT No.: PCT/FR2007/000047

§ 371 Date: Dec. 23, 2008

(30) Foreign Application Priority Data
Jan. 12, 2006 (FR) 0600258

(51) Int. Cl. A61K 31/215; A61P 31/16; A61K 9/48

(52) U.S. Cl. 424/451; 514/529

(57) ABSTRACT

The invention relates to a method for manufacturing a pharmaceutical form of oseltamivir phosphate, characterized by including the following steps:
a) compacting followed by calibration of an oseltamivir phosphate powder,
b) dry mixing with the known product excipients obtained from the previous step, following by calibration.

Application to industrial production adapted to a crisis situation such as a pandemic.

The invention relates to an industrial production process for densified oseltamivir phosphate.

In particular, the invention relates to a method for manufacturing a pharmaceutical form of oseltamivir phosphate suitable for a crisis situation when large quantities of product must be reproducibly and reliably manufactured in a short space of time such as during a pandemic.

Oseltamivir phosphate is an antiviral agent used for the prevention and treatment of influenza.

Oseltamivir phosphate is manufactured and sold under the trade name Tamiflu® by the Hoffmann-La Roche Company. This drug is available on the market in the form of 75 mg gel capsules for adult dosing and in the form of a suspension for reconstitution in the powder form yielding infant, pediatric, and adolescent dosages of 30 mg, 45 mg, and 60 mg. The dosage depends on the body weight and age of the patient.

The dosage for influenza treatment is two doses a day, for each dosage level, for five days.

Influenza pandemics are exceptional events that can rapidly involve almost every country in the world.

Pandemics are due to rapid propagation, in humans, of a virus by coughing or sneezing; moreover, the infected subjects can excrete viruses before symptoms appear, which aggravates the risk of international propagation by travelers.

The World Health Organization (WHO) has identified a risk of avian influenza.

Avian influenza is a contagious disease that affects animals. It is caused by viruses that normally infect only birds and, more rarely, swine. Influenza viruses are highly species-specific but, on rare occasions, have crossed the species barrier to humans.

Influenza viruses are divided into 3 types: A, B, and C. Only influenza viruses A can cause pandemics. Influenza viruses A have 16 H subtypes and 9 N subtypes. Only the H5 and H7 subtypes of the virus can be highly pathogenic. The H5N1 virus has proven to be especially tough. The widespread persistence of the H5N1 virus in poultry populations constitutes a risk for human health. Among the few avian influenza viruses that have crossed the species barrier and infected humans, the H5N1 virus is one that has caused the largest number of serious and fatal cases.

Two drugs belonging to the neuraminidase inhibitor class, oseltamivir (propriety name Tamiflu®) and zanamivir (proprietary name Relenza®) reduce the duration of seasonal influenza.

Neuraminidase inhibitors are effective provided they are administered within 48 hours of the onset of symptoms. In the case of human infection, these drugs can improve the prospects for survival if administered quickly. The H5N1 virus should be susceptible to neuraminidase.

The article by Penelope Ward and al. of the Hoffmann-La Roche Company in the Journal of Antimicrobial Chemotherapy (2005), Vol. 55, Suppl. S1, pp. i5-i21, entitled “Oseltamivir (Tamiflu®) and its potential use in the event of an influenza pandemic” states that oseltamivir is active in the treatment and prevention of avian influenza. The main constraints to the implementation of neuraminidase inhibitors relate to the limited production capacity and a price that is prohibitive for many countries.

The WHO has recommended that countries with sufficient resources stockpile antivirals at the national level for the start of a pandemic.

Oseltamivir is the international nonproprietary name (INN) of 3R,4R,5S)-4-acetylamino-5-amino-3(1-ethylpropoxy)-1-cyclohexene-1-carboxylic acid ethyl ester and its pharmaceutically acceptable salts of addition such as phosphate.

The manufacturing process for the drug developed by the Hoffmann-La Roche Company, wet granulation followed by drying, is necessary for densification of the active ingredient before the pharmaceutical forms are produced. This process is described in US Patent Application 2002/0018812 in the name of the Hoffmann-La Roche Company.

This manufacturing process has a number of drawbacks in the event of a pandemic.

The two drugs from the Hoffmann-La Roche Company are the 75 mg capsules for adults and the suspension for reconstitution in the powder form yielding infant, pediatric, and adolescent dosages of 30 mg, 45 mg, and 60 mg.

Manufacturing the 30 mg, 45 mg, and 60 mg dosages involves a complex formulation in a glass flask of which the production rates, in the pharmaceutical industry in general, are not appropriate for a pandemic context.

The pharmaceutical forms of the Hoffmann-La Roche Company require large-scale stockpiling of the containers required for their manufacture such as capsules and glass flasks, as well as excipient inventories.

Moreover, the percentage of each age group susceptible to the virus cannot be predicted, so management of the two forms in difficult in both logistical and in financial terms.

Finally, the actual start of a human avian influenza pandemic cannot be known in advance, [and] even though it is considered inevitable by the WHO, the purchase of an inventory in the form of Hoffmann-La Roche Company drugs represents a considerable financial outlay. Moreover, the inventory would have to be refreshed according to the expiration dates of the drugs stored.

In addition, the inventory would have to be inflated to ensure that a sufficient amount would be available for each form.

In the context of this public health project piloted by the General Health Department (DGS), the Army Central Pharmacy (PCA) was contacted, as were other public and private laboratories, to set up large-scale manufacturing of one or more pharmaceutical forms suitable for a pandemic, starting from the active ingredient oseltamivir phosphate manufactured by the Hoffmann-La Roche Company.

Once a sample of oseltamivir phosphate had been received by the PCA, the pharmacotechnical tests performed showed that it was impossible to use this active ingredient in an industrial process without prior modification, due to the exceptionally low density for a pharmaceutical material and natural clinging to the equipment, particularly because of electrostatic characteristics.

In order to remedy the afore-mentioned drawbacks, the goal of the invention, in the context of a crisis situation such as a pandemic, is to arrive at industrial production of densified oseltamivir phosphate and from it to manufacture tablets, capsules, or powder packets. The idea was conceived of using wet granulation to obtain an appropriate process for industrial production and/or stockpiling on a large scale, but the physical properties of oseltamivir do not enable the individual skilled in the art to envisage such a technique.

To accomplish this, the invention relates to a method for manufacturing a pharmaceutical form of oseltamivir phosphate, characterized by including the following steps:

a) compacting followed by calibration of an oseltamivir phosphate powder,

b) dry mixing with the known product excipients obtained from the previous step, following by calibration.

Manufacturing is performed continuously with the aid of a compactor equipped with its in-line calibrator.

The compacted, calibrated oseltamivir phosphate obtained from step a) is stored and step b) can be performed later.

According to one embodiment, the oseltamivir phosphate is combined with a diluent, a binder, a disaggregant, a flow lubricant, and an anti-adherent.

Advantageously, the diluent and binder is comprised of high-density microcrystalline cellulose known under the Avicel trade names. Avicel pH 102, Avicel pH 112, Avicel pH 200, and Avicel pH 302 have been tested successfully.

The disaggregant is sodium croscarmellose known under the trade name Ac-Di-Sol.

The flow lubricant is colloidal silica known under the trade name Aerosil 200.

The lubricant and anti-adherent is sodium stearyl fumarate.

According to one embodiment, the product obtained is converted either into a tablet by direct compression or into a capsule by automatic filling of a capsule, or into a single-dose powder packet.

Preferably, the tablet is a 150 mg scored tablet containing 30 mg basic oseltamivir.

The oseltamivir phosphate crystallizes in the form of needle-shaped fibrous particles.

Because of the shape of the crystal, the active ingredient has a plushy appearance and has very low density.

These features imply special treatment during production of the pharmaceutical form which requires granulation in order to keep a mixture of independent particles homogenous.

The manufacturer chose a wet granulation method to ensure the homogeneity of the content and easy large-scale manufacturing.

The wet granulation method involves mixing the excipients with the active ingredient, adding a wetting solution (with or without binder), and bringing about granulation. This step is followed by drying which allows densification, then calibration of the dry granulate, screening, and final mixing.

Wet granulation is the most commonly used process for obtaining the formation of relatively porous solid agglomerates whose physical properties provide the initial powder mix with better homogeneity and optimal flow, without unmixing, for unit filling into capsules or packets and better cohesion of the tablet form.

The lattice network thus created can also favor subsequent dissolution of the product in water or in the body, and improve its bioavailability.

However, wet granulation is a lengthier and more-complex method to implement than mere mixing of the active ingredient with the particular excipients or dry granulation which uses only a mechanical operation.

It involves more steps in the operation and special equipment.

In the presence case, the active ingredient poses various production difficulties at the outset because its density is approximately 0.1 g/cm³, it does not flow, and without densification it is impossible to fill the volume represented by the necessary dose into a capsule.

Moreover, the powder is highly aerophilic so that it cannot be produced at a high rate.

In view of the aforesaid difficulties, wet granulation was chosen.

This method uses a compacting technique.

Compacting consists of forcing the powder between two cylindrical, parallel rollers rotating in opposite directions. As the volume decreases in the zone of maximum compression; the material takes on the form of a compact solid.

The compacting process is governed by factors such as the surface area, diameter, and peripheral speed of the rollers, the compressive force, the design of the feed system, and the characteristics of the actual material to be compacted.

When the material has a low density, compacting may be difficult to accomplish because of air retention inside the material which offers resistance to the pressure applied by the rollers. This may be remedied by introducing a vacuum source at the material feed.

The compacted product needs to be calibrated to a uniform particle-size distribution. This operation is effected by an oscillating calibrator.

Compacting has the advantage that the operation can be continuous so that the yields are far higher with wet granulation for a given size.

However, the internal cohesion of the grains remains lower and not all the products are compatible.

In the present case, substantial difficulties arose in the compacting tests on the initial oseltamivir phosphate mixes with excipients and pure product.

The particular characteristics of oseltamivir phosphate, such as the absence of powder fluidity so that there is zero flow, its very low density, its ability to stick to the equipment walls by static electricity, and its tendency to stick to the equipment if pressure is applied, have caused agglutination of the pure product or mix on the walls of the feed screw. The result was highly irregular or zero feed into the compacting rollers. Consequently the product was insufficiently compacted and its granulometric properties, essential for the remainder of the manufacturing process, could not be defined.

These technical difficulties meant that compacting had to be abandoned. However, they were resolved by the non-obvious choice of the best material and compacting parameter compromises.

With regard to the materials, a choice had to be made between stainless steel, electropolished, and teflonized stainless steel, particularly for the precompacting screws and sheath or cylinder, and the grooving of the compacting rollers had to be designed so that they would “catch” the product and evacuate it in the form of properly shaped platelets that are densified at a constant rate before in-line calibration.

Concerning the compacting parameters: the feed screw speed, the speed of the compacting rollers, the pressure between the rollers, and the mesh size of the calibration grid of the in-line calibrator has to be determined.

The description will be better understood with the aid of the embodiment examples described below.

### COMPACTING EXAMPLES

Feasibility tests were performed on a compactor designed for pharmaceutical use, known by its trade name Hosokawa Bepex Pharmapaktor L200/50P.

Various parameters were tested for compacting pure oseltamivir phosphate.

The table below shows various tests that enabled the optimized compacting parameters to be determined.

| | | | | | | Product |
|---|---|---|---|---|---|---|
| | | | | Pressure | Mesh | density |
| Test | Rollers | Screw | Sheath | (kN) | (mm) | (g/cm3) |
| 1 | Flat lozenges | Electro- | Crenellated | 14 | 1 | 0.35 |
| | | polished | stainless steel | | | |
| | | stainless steel | | | | |
| 2 | Flat lozenges | Teflon | Crenellated | 14 | 1 | 0.35 |
| | | | stainless steel | | | |
| 3 | Flat lozenges | Teflon | Teflon | 15 | 1 | 0.32 |
| 4 | Straight concave | Teflon | Teflon | 16 | 1 | 0.36 |
| | grooves | | | | | |
| 5 | Straight concave | Electro- | Teflon | 15 | 1 | 0.35 |
| | grooves | polished | | | | |
| | | stainless steel | | | | |
| 6 | Straight concave | Electro- | Teflon | 15 | 0.8 | 0.40 |
| | grooves | polished | | | | |
| | | stainless steel | | | | |

Preferably, all the equipment in contact with the product is made of electropolished stainless steel.

It can be seen from the table that the combination of parameters in Tests 1 to 3 is not adequate. Moreover, it does not enable a constant and sufficient production rate to be achieved.

A free-flowing densified product with a density of 0.35 a 0.45 g/cm³ was obtained, with good direct compressibility and compatible with filling in capsules.

### Examples of Pharmaceutical Forms:

The following excipients were used:

Microcrystalline cellulose known under the trade name Avicel pH 302 as a diluent and binder,

sodium croscarmellose known under the trade name Ac-Di-Sol as a disaggregant,

colloidal silica known under the trade name Aerosil 200 as a flow lubricant

sodium stearyl fumarate as an anti-adherent.

Following tests using various types of microcrystalline cellulose known under the trade name Avicel, a choice was made of Avicel pH 302 to obtain better flow of the mix so that higher production rates could be expected. As an example, the following proportions can be used.

| | Proportion |
|---|---|
| Material | (percentage by mass) |
| Oseltamivir phosphate | Up to 50 |
| Sodium stearyl fumarate | 0.5 to 2 |
| Colloidal silica (Aerosil 200) | 0.25 to 1 |
| Sodium croscarmellose (Ac-Di-Sol) | 1 to 10 |
| Microcrystalline cellulose | qsp. 100 |
| (Avicel pH 302) | |

### Production Example of a Scored Tablet:

The pharmaceutical form is a tablet 8 mm in diameter and 16 mm in radius of curvature that can be broken in half and covers the dosage range recommended for treatment of human influenza by Tamiflu®, namely 30, 45, 60, and 75 mg.

This tablet, with a weight of 150 mg, contains 30 mg of basic oseltamivir, i.e. 39.4 mg oseltamivir phosphate and, as excipients, Avicel® pH 302, Ac-Di-Sol®, Aerosil® 200, and sodium stearyl fumarate.

The solution of compacting a mixture of oseltamivir phosphate and excipients was abandoned in favor of compacting only the active ingredient.

This option has the advantage of allowing both compacting and calibration of the pure oseltamivir phosphate in advance, which does not affect the stability of the active ingredient over time and allows the active ingredient to be stored so that it is ready to use.

Thus, pure, compacted, calibrated oseltamivir phosphate can be stockpiled until there is a demand for producing the drug.

Only then will the mixing necessary for making tablets, capsules, and powder packets be done.

The method according to the invention reduces the manufacturing time of the pharmaceutical form.

1. A method for manufacturing a pharmaceutical form of oseltamivir phosphate, including the following steps:
a) compacting an oseltamivir phosphate powder, followed by calibration of the oseltamivir phosphate powder,
b) dry mixing the oseltamivir phosphate powder with known product excipients obtained from step a), followed by another calibration.

2. The method according to claim 1, wherein the manufacturing is performed continuously with a compactor equipped with an in-line calibrator.

3. The method according to claim 1, wherein the compacted, calibrated oseltamivir phosphate obtained from step a) is first stored, and step b) is performed later on demand.

4. The method according to claim 1, wherein the oseltamivir phosphate is combined with excipients including a diluent, a binder, a disaggregant, a flow lubricant, and an anti-adherent.

5. The method according to claim 4, wherein the diluent and the binder are comprised of high-density microcrystalline cellulose.

6. The method according to claim 4, wherein the disaggregant is sodium croscarmellos.

7. The method according to claim 4, wherein the flow lubricant is colloidal silica.

8. The method according to claim 4, wherein the flow lubricant and the anti-adherent are sodium stearyl fumarate.

9. The method according to claim 1, wherein the oseltamivir phosphate powder obtained is converted into at least one of a tablet by direct compression, a capsule by automatic filling of a capsule, and a single-dose powder packet.

10. The method according to claim 9, wherein the oseltamivir phosphate powder obtained is converted into a 150 mg scored tablet containing 30 mg basic oseltamivir.

11. The method according to claim 2, wherein the compacted, calibrated oseltamivir phosphate obtained from step a) is first stored, and step b) is performed later on demand.
